(19) United States
(12) Patent Application Publication
(10) Pub. No.: US 2004/0038221 A1
(43) Pub. Date: Feb. 26, 2004

(54) Test system for detecting a splicing reaction and use therof

(76) Inventors: Bettina Bauer, Frankfurt (DE); Eberhard Schneider, Kelkheim (DE); Marion Bartel, Leipzig (DE)

Correspondence Address: CONNOLLY BOVE LODGE & HUTZ, LLP, P O BOX 2207, WILMINGTON, DE, 19899, US

(21) Appl. No.: 10/257,554

(22) PCT Filed: Feb. 28, 2001
(86) PCT No.: PCT/EP01/02234

(30) Foreign Application Priority Data
Apr. 14, 2000 (DE) 100 18 465.0

(51) Int. Cl. C12Q 1/68

(52) U.S. Cl. 435/6

(57) ABSTRACT

The invention relates to a homogenous test system containing the following: (a) at least one or more, identical or different mobile nucleic acids with at least one spliceable nucleic acid, containing at least one intron; (b) at least two probes, containing corresponding fluorophores which preferably bind with the 5′ and/or 3′ ends of an exon/intron; (c) at least one gel-free detecting system for detecting a splicing reaction; and optionally (d) at least one composition containing splicing components; and (e) other auxiliary agents.

### DESCRIPTION

The present invention relates to a homogeneous test system comprising

(a) at least one or more identical or different mobile nucleic acid(s) having at least one spliceable nucleic acid comprising at least one intron,

(b) at least two probes, comprising corresponding fluorophores which preferably bind to the 5′ and/or 3′ ends of an exon/intron,

(c) at least one gel-free detection system for detecting a splicing reaction, where appropriate

(d) at least one composition comprising splicing components, and preferably

(e) further aids.

Most of the protein-encoding genes in eukaryotes are interrupted in their genomic form by one or more sequences not coding for the protein (introns). When transcribing the genomic DNA into messenger RNA (mRNA), these non-coding regions (introns) are incorporated into the primary transcript. In order to generate the correct mRNA, this pre-mRNA has to be processed.

The pre-mRNA is processed by removing the introns and fusion of the coding regions (exons). Only then is it possible to provide a nucleotide strand which can be read in an interrupted manner for translation in the cytoplasm. mRNA formation in eukaryotes therefore requires a “splicing process” in which the non-coding gene regions (introns) are removed from the primary gene transcript.

Splicing occurs in the nucleus, before the mRNA is transported out of the nucleus. It is generally carried out in a two-stage mechanism in which in each case a transesterification step is involved (Moore, J. M. et al., (1993) Splicing of precursors to mesenger RNAs by the Spliceosome. In The RNA world, Edited by Gesteland R. F., Gesteland, J. F., Cold Spring Harbor Laboratory Press, 303-358). The first step generates a free 5′ exon and a “lariat structure” of the intron which is still linked to the 3′ exon. The lariat structure comprises a branched RNA which is produced by esterification of the 5′ end of the intron with a 2′-hydroxyl group of a ribose in an adenosine which is located approx. 20-40 nucleotides upstream of the 3′ end of the intron, the so-called branch site. The second catalytic step leads to ligation of the exons and liberation of the intron. Although no nucleotides are incorporated during these reactions, an energy source, for example ATP, is necessary for this catalysis (Guthrie, C. (1991) Science, 253,157).

A plurality of factors is involved in the process of mRNA splicing. Two classes of splicing factors are distinguished at the moment. The first class comprises four evolutionarily highly conserved protein-RNA particles (small nuclear ribonucleoprotein particles=snRNPs); U1, U2, U4/U6 and U5, which comprise either one (U1, U2, U5) or two (U4/U6) snRNA components (Moore, J. M. et al., (1993) supra; Guthrie, (1991) supra; Green, M. R. (1991), Annu. Rev. Cell Biol., 7, 559). The second class comprises proteins which have not been characterized much up until now and which are not tightly bound to the snRNPs and are therefore called non-snRNP splicing factors (Lamm, G. M. & Lamond, A. J. (1993) Biochim. Biophys. Acta, 1173, 247; Beggs, J. D. (1995), Yeast splicing factors and genetic strategies for their analysis, In: Lamond, A. I. (ed) Pre-mRNA Processing Landes, R. G. Company, Texas, pp. 79-95. Krämer A. (1995), The biochemistry of pre-mRNA splicing. In: Lamond, A. I. (ed), Pre-mRNA Processing. Landes, R. G. Company, Texas, pp. 35-64).

The composition of snRNPs has been studied most successfully in HeLa cells (Will, C. L. et al., (1995) Nuclear pre-mRNA splicing. In: Eckstein, F. and Lilley, D. M. J. (eds). Nucleic Acids and Molecular Biology. Springer Verlag, Berlin, pp. 342-372). At relatively low salt concentrations at which it is possible for nuclear extracts from HeLa cells to promote splicing of pre-mRNA in vitro, the snRNPs are present in a 12S U1 snRNP, a 17S U2 snRNP and a 25S [U4/U6.U5] tri-snRNP complex. At higher salt concentrations (approx. 350-450 mM) the tri-snRNP complex dissociates into a 20S U5 particle and a 12S U4/U6 particle. In the U4/U6 snRNP, the U4 and U6 RNAs are present base-paired via two intermolecular helices (Bringmann, P. et al. (1984) EMBO J., 3,1357; Hashimoto, C. & Steitz, J. A. (1984) Nucleic Acids Res., 12, 3283; Rinke, J. et al., (1985) J. Mol. Biol., 185, 721; Brow. D. A. & Guthrie, C. (1988) Nature, 334,213).

The snRNPs comprise two groups of proteins. All snRNPs comprise the group of general proteins (B/B′, D1, D2, D3, E, F and G). In addition, each snRNP comprises specific proteins which are present only in said snRNP. Thus, according to the prior state of research, U1 snRNP comprises three additional proteins (70K, A and C) and U2 snRNP eleven further proteins. According to prior knowledge, 20S U5 snRNP carries nine further proteins having molecular weights of 15, 40, 52, 100, 102, 110, 116, 200 and 220 kDa, while 12S U4/U6 snRNP comprises two additional proteins having molecular weights of approx. 60 and 90 kDa. 25S tri-snRNP [U4/U6.U5] comprises five additional proteins having molecular weights of approx. 15.5, 20, 27, 61 and 63 kDa (Behrens, S. E. & Lührmann, R. (1991) Genes Dev., 5, 1439; Utans, U. et al., (1992) Genes Dev., 6, 631; Lauber, J. et al., (1996) EMBO J., 15, 4001; Will, C. L. et al. (1995), supra, Will, C. L. & Lührmann, R. (1997) Curr. Opin. Cell Biol., 9, 320-328).

The composition of splicing components in Saccharomyces cerevisiae has not yet been studied in detail. Biochemical and genetic studies, however, indicate that the sequences of both the snRNAs and the snRNP proteins are evolutionarily highly conserved (Fabrizio, P. et al., (1994) Science, 264, 261; Lauber, J. et al., (1996), supra, Neubauer, G. et al., (1997) Proc. Natl. Acad. Sci. USA, 94, 385; Krämer, A. (1995), supra; Beggs, J. D. (1995); supra, Gottschalk, A. et al. (1998) RNA, 4, 374-393).

In order to form a functional splicing complex (spliceosome), the individual components (pre-mRNA, snRNPs and non-snRNP proteins) are combined in a stage-wise process. This is achieved not only by interactions of the pre-mRNA with the protein-containing components but also by numerous interactions between the protein-containing components themselves (Moore, J. M. (1993) supra; Madhani, H. D. & Guthrie, C. (1994) Annu. Rev. Genetics, 28, 1; Nilsen, T. W. (1994) Cell, 65, 115). The pre-mRNA sequence carries specific recognition sequences for the different splicing components. Firstly, U1 snRNP binds via said recognition sequences to the 5′ splicing region of the pre-mRNA intron. At the same time, an as yet unspecified number of various other factors (e.g. SF2/ASF, U2AF, SC35, SF1) is taken up by this complex and cooperates with the snRNAs in the continued formation of the pre-spliceosome. The U2 snRNP particle interacts with the “branch site” in the intron region (Kr\overline{a}mer, A. & Utans, U. (1991) EMBO J., 10, 1503; Fu, X. D. & Maniatis, T. (1992) Proc. Natl. Acad. Sci USA, 89, 1725; Krämer, A. (1992) Mol. Cell Biol., 12, 4545; Zamore, P. D. et al. (1992) Nature, 355, 609; Eperon, J. C. et al. (1993) EMBO J., 12, 3607; Zuo, P. (1994) Proc. Natl. Acad. Sci. USA, 91, 3363; Hodges, P. E. & Beggs, J. D. (1994) Curr. Biol. 4, 264; Reed, R. (1996) Curr. Op. Gen. Dev., 6, 215). In a last step of spliceosome formation, [U4/U6.U5] tri-snRNP and a number of proteins not yet characterized in detail interact with the pre-spliceosome, in order to form the mature spliceosome (Moore, J. M. et al., (1993) supra).

For the splicing process, various interactions between pre-mRNA, snRNAs and sn-RNP are removed and new ones are formed. Thus it is known that before or during the first catalytic step of the splicing reaction two helices are separated from one another in the interacting structures of U4 and U6 and that new interactions form base pairs between U2 RNAs and U6 RNAs (Datta, B. & Weiner, A. M. (1991) Nature, 352, 821; Wu, J. A. & Manley, J. L. (1991) Nature, 352, 818; Madhani, H. D. & Guthrie, C. (1992) Cell, 71, 803; Sun, J. S. & Manley, J. L. (1995) Genes Dev., 9, 843). At the same time, binding of U1 to the 5′ splicing site is removed and pre-mRNA binds to the recognition sequence ACAGAG of U6 snRNA (Fabrizio, P. & Abelson, J. (1990), Science, 250, 404; Sawa, H. & Abelson, J. (1992) Proc. Natl. Acad. Sci. USA, 89, 11269; Kandels-Lewis, S, & Seraphin, B. (1993) Science, 262, 2035; Lesser, C. F. & Guthrie C. (1993) Science, 262, 1982; Sontheimer, E. J. & Steitz, J. A. (1993) Science, 262, 1989). U5 snRNP interacts via its conserved loop 1 with exon sequences which are located close to the 5′ and 3′ splicing sites. This process seems to be sequential, while the entire splicing process progresses from stage 1 to stage 2 (M. McKeown (1992) Annu. Rev. Cell Dev. Biol., 8: 133-155 Newman, A. & Norman, C. (1991) Cell, 65, 115; Wyatt, J. R. et al. (1992) Genes Dev., 6, 2542; Cortes, J. J. et al. (1993) EMBO J., 12, 5181; Sontheimer, E. J. & Steits (1993) supra). After the splicing reaction has finished, the mature mRNA is liberated and the spliceosome dissociates (Moore, J. M. et al (1993) supra).

Alternative splicing makes it possible to form from one and the same primary transcript various mature mRNAs which code for various proteins. In many cases, this alternative splicing is regulated. Thus it is possible to utilize this mechanism, for example, for the purpose of switching from a non-functional to a functional protein (e.g. transposase in Drosophila). It is furthermore known that alternative splicing is carried out tissue-specifically. Thus, for example, tyrosine kinase which is encoded by the src proto-oncogene is synthesized in nerve cells in a specific form by alternative splicing.

Incorrectly regulated or performed alternative splicing may lead to various conditions. In patients suffering from Graves' disease it has been shown that incorrect splicing produces a crucial enzyme (thyroperoxidase) in an inactive form (Zanelli, E. (1990) Biochem. Biophys. Res. Comm., 170, 725). Studies of the disease spinal muscular atrophy indicate that a defective gene product of the SMN (survival of motor neurons) gene leads to a considerable disruption in the formation of snRNPs. Inhibition of the splicing apparatus of the motor neurons leads to paralysis of the nerve cells and to degeneration of muscle tissue (Fischer, U. et al. (1997), Cell, 90: 1023-9; Liu, Q. et al. (1997), Cell, 90: 1013-21; Lefebvre, S. et al. (1997) Nat. Genet. 16, 265). Particular alternative splicing variants of the membrane-bound molecule CD44, inter alia, seem to play a decisive part in cancer cell metastasis. The CD44 gene comprises a plurality of exons, 10 exons of which, located next to one another, are spliced from the pre-mRNA in different arrangement during mRNA generation. In rat carcinoma cells it was detected that metastasizing variants carry exons 4 to 7 or 6 to 7. With the aid of antibodies against the exon 6-encoded part of the protein it was possible to suppress metastasis efficiently (Sherman, L., et al. (1996) Curr. Top. Microbiol. Immunol. 213: 249-269).

Splicing mutants of the APC (adenomatous poliposis coli) oncogene, which have been isolated from patients with familiar adenomatous poliposis (FAP), produce truncated proteins due to incorrect alternative splicing, with exons 9, 10A and 14 being excised (Bala, S., et al., (1996) Hum Genet 98(5): 528-533).

Incorrect splicing may lead to strongly developed phenotypes of the affected organism. Thus it is known that a point mutation in a β-globin intron may lead to a β⁺ thalassemia.—The point mutation produces an incorrect splicing location which leads to a modified reading frame and to preliminary termination of the peptide chain (Weatherall, D. J. & Clegg, J. B. (1982) Cell, 29, 7; Fukumaki, Y. et al. (1982) Cell, 28, 585). In Arabidopsis thaliana mutants, for example, a point mutation at the 5′ splicing site of the phytochrome B gene leads to incorrect expression of the gene. This modification makes it impossible to remove an intron whose sequence includes a stop codon. Development of the plants is disrupted, since the gene is involved in phytomorphogenesis (Bradley, J. M. et al. (1995) Plant Mol. Biol., 27,1133).

Up until now, only a few studies have been known, which have described influencing of splicing processes in the cell. Thus it is possible, with the aid of antisera or monoclonal antibodies against components of the splicing apparatus, to prevent generation of mature mRNA (Padgett, R. A. et al. (1983) Cell, 35,10; Gattoni, R. et al. (1996) Nucleic Acid Res., 24, 2535).

The NS1 protein which is encoded by the influenza virus genome may likewise interfere in splicing by binding to U6 snRNA. The protein binds to nucleotides 27-46 and 83-101 of human U6 snRNA and thus prevents U6 from being able to interact with the partners U2 and U4 during the splicing process (Fortes, P. et al. (1994) EMBO J., 13, 704; Qiu, Y. & Krug, R. M. (1995) J. Virol., 68, 2425). Moreover, the NS1 protein also seems to prevent export from the nucleus by binding to the polyA tail of the mRNA formed (Fortes, P. et al. (1994), supra; Qiu, Y. & Krug, R. M. (1994), supra). Similar actions are described for a gene product of the Herpes simplex virus type 1 genome. In in vitro experiments, the protein ICP27 was able to effectively prevent splicing of a model RNA (β-globin pre-mRNA) (Hardy, W. R. & Sandri-Goldin, R. M. (1994) J. Virol., 68, 7790). In addition, peptides which have been generated from the C-terminal domain of the large subunit of RNA polymerase II seem likewise to be able to interfere in splicing processes (Yurvey, A. et al. (1996) Proc. Natl. Acad. Sci USA, 93, 6975; WO97/20031). The incorporation of artificial nucleotide analogs (5-fluoro-, 5-chloro- or 5-bromouridine) into the mRNA to be spliced may likewise lead to inhibition of the splicing process in vitro (Sierakowska, H. et al. (1989) J. Biol. Chem., 264, 19185; Wu, X. P. & Dolnick, B. (1993) Mol. Pharmacol., 44, 22).

A number of further studies relates to the action of antisense oligonucleotides on splicing. Thus, the ratio of two different splicing products of the c-erb oncogene mRNA (c-erbA-alpha 1 and 2) from rats seems to be regulated by another mRNA, rev-ErbA-alpha. Rev-ErbA-alpha is a naturally occurring antisense RNA which pairs with c-erbA-alpha 2 mRNA but not with c-erbA-alpha 1 mRNA. An excess of rev-ErbA-alpha mRNa constructs which were complementary to the 3′ splicing site made it possible to effectively inhibit splicing of c-erbA-alpha pre-mRNA to c-erbA-alpha 2 mRNA (Munroe, S. H. & Lazar, M. A. (1991) J. Biol. Chem. 266 (33), 22083). Furthermore it was shown that generation of antisense RNA which bind to intron sequences of the mRNA to be spliced may likewise inhibit splicing (Volloch, V. et al. (1991) Biochem. Biophys. Res. Comm., 179, 1600). Hodges and Crooke were able to show that for weakly recognized splicing sites oligonucleotide binding is sufficient in order successfully to stop splicing. If, however, preferably recognized splicing sites are incorporated into the constructs, oligonucleotides which in addition can cause activation of RNase H are required (Hodges, D. & Crooke S. T. (1995) Mol. Pharmacol., 48, 905). A more detailed analysis of the pre-mRNA sequences required for splicing showed that 19 nucleotides upstream from the branching point adenosine and 25 nucleotides around the 3′ and 5′ splicing site are suitable sequences for generating antisense RNAs (Dominski, Z. & Kole, R. (1994) Mol. Cell Biol., 14, 7445). Studies with antisense molecules were carried out in particular for inhibition of viruses. Viruses which effect higher organisms often carry intron-containing genes in their genome. Thus it was possible to show that antisense oligonucleotides against the 3′ splicing site of the immediate early pre-mRNA 4/5 gene of Herpes simplex virus was able to inhibit virus replication in Vero cells (Iwatani, W. et al. (1996) Drug Delivery Syst., 11, 427).

The splicing mechanism is studied in general firstly by preparing pre-mRNA by in vitro transcription. To this end, genetic constructs from viruses, for example adenoviruses, or cellular structural genes are used. Pre-mRNAs of this kind include all important structural elements which are necessary for recognition of the pre-mRNA by the spliceosome and for the splicing process. Generally, the pre-mRNA is radiolabelled in order to make it possible, after fractionation in a denaturing urea polyacrylamide gel, to evaluate, owing to the characteristic band pattern, whether a splicing reaction has occurred or in which reaction step a disruption has occurred. However, test systems of this kind are very time-consuming and labor-intensive and are therefore not suited to the systematic finding of substances which can modulate splicing.

The applicant's earlier German patent application 199 09 156 discloses an in vitro splicing assay which makes it possible to study in a simple and effective manner a large number of compounds from chemical or natural substance libraries for their action on splicing of nucleic acids (high throughput screening). However, the spliceable nucleic acid have to be immobilized in this case. This is therefore a heterogeneous system which requires various washing steps.

It was therefore an object of the present invention to find a homogeneous test system which makes it possible to study in a simple and effective manner a large number of compounds from chemical or natural substance libraries for their action on splicing of nucleic acids (high throughput screening).

Surprisingly, it has now been found that a homogeneous test system with a gel-free detection system with specific probes, in which system all components are mobile in a soluble phase, for detecting a splicing reaction is suitable to overcome the above-described disadvantages of the conventional test systems and is thus suitable for homogeneous high throughput screening, for example in a robot system.

The present invention therefore relates to a homogeneous test system comprising

(a) at least one or more identical or different mobile nucleic acid(s) having at least one spliceable nucleic acid comprising at least one intron,

(b) at least two probes, comprising corresponding fluorophores which preferably bind to the 5′ and/or 3′ ends of an exxon/intron,

(c) at least one gel-free detection system for detecting a splicing reaction, where appropriate

(d) at least one composition comprising splicing components,

(e) further aids.

Therefore, suitable probes which make it possible to detect the splicing which has or has not occurred are to be generated for a homogeneous test system. The probes according to (b) can bind to the nucleic acid to be studied, preferably by means of hybridization.

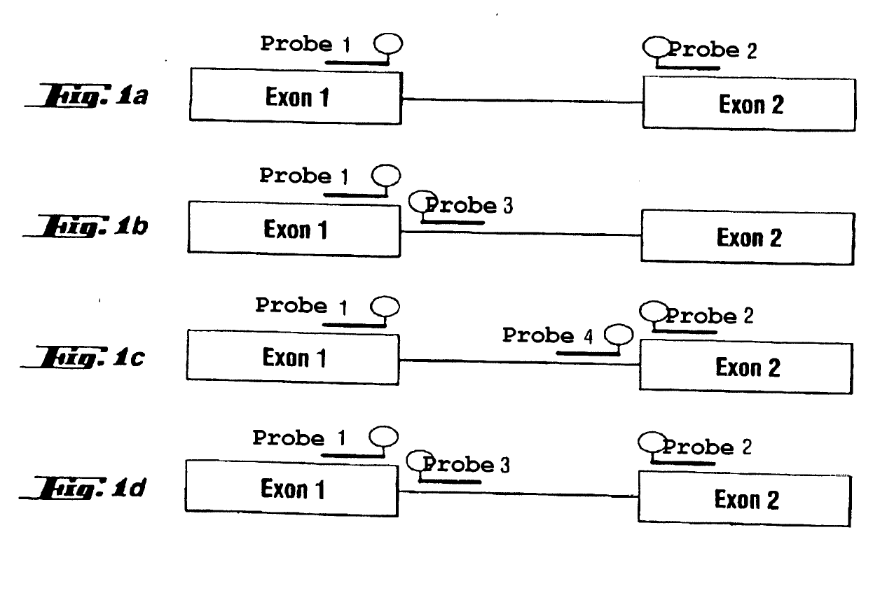

The nucleic acids of such probes, which are required for this and which are complementary to partial sequences of the spliceable nucleic acid, are complementary to at least one partial sequence of an intron and one partial sequence of an exon or to at least two partial sequences of two exons. The arrangement in principle is shown in FIGS. 1a)-d).

The complementary probes are preferably located at the ends of the intron or exon and, owing to corresponding fluorophores in the unspliced, spliceable nucleic acid or in the splicing product, generate a detectable signal which makes it possible to characterize and study the state of maturity of the RNA (pre-mRNA or mRNA).

The term “fluorophore” indicates to the skilled worker molecules whose physical behavior is characterized by the fact that they are excited, i.e. elevated to a higher energy level, by a distinct wavelength and that they emit this additional energy in the form of light of longer wavelength when they return to their original energy level.

Corresponding fluorophores in accordance with the invention means a pair of fluorescent molecules whose properties comprise the fact that the wavelength of the emitted light (emission) of the fluorophore of lower wavelength (donor) is in the range of the excitation wavelength of the second fluorophore (acceptor). Examples of natural fluorophore pairs are phenylalanine/tyrosine and tyrosine/tryptophan in proteins. According to the invention, preference is given to corresponding fluorophores based on fluorescein/rhodamine and derivatives therefrom such as carboxyfluorescein succinimidyl ester/Texas Red sulfonyl chloride, FITC/TMR (fluorescein-5-isothiocyanate/tetramethylrhodamine, and naphthalene/dansyl, dansyl/DDPM (N-[4-dimethylamino)-3,5-dinitrophenyl]maleimide), IAEDANS/DiO-C14 (5-(2-((iodoacetyl)amino)ethyl)amino)naphthalene-1-sulfonic acid/3,3-ditetradodecyloxacarbocyanine), ANAI/IPM (2-anthracene-N-acetylimidazole/3-(4-isothiocyanatophenyl)-7-diethyl-4-amino-4-methylcoumarin, BPE/CY5 (B-phycoerythrin/carboxymethyl indocyanine N-hydroxysuccimidyl ester) and others all of which are commercially available.

However, very particular preference is given to corresponding fluorophores based on lanthanide chelates, likewise corresponding to fluorescein, which are disclosed in the patent applications WO 97/29373 and 98/15380 and are commercially available (Wallac, Turku, Finland).

The fluorescent molecules can be linked to the nucleotides during synthesis of a nucleotide sequence or else covalently connected specifically to the 5′ or 3′ terminus of the nucleotide sequence by using methods known to the skilled worker (see examples).

Moreover, preference is given to those corresponding fluorophores which are accessible to the advantageous time-resolved fluorescence resonance energy transfer.

The splicing product resulting from the splicing reaction, i.e. mature mRNA, can be detected according to the invention by means of the corresponding fluorophores, as a consequence of which both substeps (in each case exon 1/intron/exon 2 cut at the characteristic consensus sequences) of a splicing reaction have been completed.

In a particularly preferred embodiment, the probes therefore comprise two oligonucleotides which are complementary to the 3′ end of exon 1 and the 5′ end of exon 2, respectively, and which in turn carry a corresponding fluorophore at their 5′ and 3′ ends, respectively. In this way it is possible to detect the splicing process which has taken place particularly easily via a signal resulting from fluorescence resonance energy transfer (cf. FIG. 1a).

In another, particularly preferred embodiment, the probe comprises two oligonucleotides which are complementary to the 3′ end of exon 1 and the 5′ end of the intron, respectively, or which are complementary to the 3′ end of the intron and the 5′ end of exon 2 and which carry a fluorescent molecule on their 5′ and 3′ ends, respectively. Thus it is possible for the splicing process to be detected particularly easily via the reduction in a signal resulting from fluorescence resonance energy transfer (cf. FIG. 1b).

In another, particularly preferred embodiment, a probe comprises two oligonucleotides which are complementary to the 3′ end of exon 1 and the 5′ end of exon 2, respectively, and which carry a fluorescent molecule on their 5′ and 3′ ends, respectively. This makes it possible, on the basis of the resulting fluorescence, to detect the splicing process which has taken place. A third oligonucleotide which is complementary to the 3′ end of the intron indicates inhibition of the splicing reaction via a corresponding signal (cf. FIG. 1c).

The oligonucleotide arrangement in FIG. 1a is used for the direct detection of a deleted intron sequence.

The oligonucleotide arrangement in FIG. 1b preferably serves to detect whether exon 1 has been separated successfully from the intron sequence during the splicing process. The oligonucleotide arrangement in FIG. 1c preferably serves to detect whether exon 2 has been separated successfully from the intron sequence.

A combination of FIG. 1a and FIG. 1b is shown in FIG. 1d and is therefore preferably used for detecting the individual intermediates and final products during the splicing process.

Therefore, the test system of the invention can be used in order to determine in which reaction step the splicing process is interrupted.

In a preferred embodiment, the spliceable nucleic acid contains at least two exons which are separated by at least one intron.

Exon 1 generally is an exon located 5′ of the intron and exon 2 generally is an exon located 3′ of the intron.

According to the present invention, the spliceable nucleic acid is any nucleic acid which can be spliced, preferably an RNA, for example in the form of a “pre-mRNA” or in the form of a DNA containing RNA sections. Intron means a noncoding sequence which is spliced owing to the recognizing consensus sequences. Such consensus sequences are in yeast: /GUAUGU in the 5′ splice site,—YAG/G in the 3′ splice site and UACUAAC in the branch point; in humans: AG/GURAGU in the 5′ splice site, YAG/ in the 3′ splice site and YNYURAC in the branch point. The underlined nucleotides are highly conserved. Very particular preference is given to an intron which is ligated with two exons terminally via consensus sequences.

An example of a spliceable nucleic acid, which is suitable for splicing in the human system, is the MINX model pre-mRNA (MINX=miniature wild type substrate; Zillmann, M., Zapp, M. L., Berget, S. M. (1998), Mol. Cell. Biol., 8:814-21).

As already mentioned, it is possible to insert additional probes into the pre-mRNA intron structure. Constructs of this kind are therefore suitable for detecting an inhibition of splicing in the first step, i.e. opening of mRNA and lariat formation, or in the second step, i.e. removal of the lariat. In the case of an inhibition of the splicing process, the fluorophores would not be separated from one another and, therefore, continue to give off a signal.

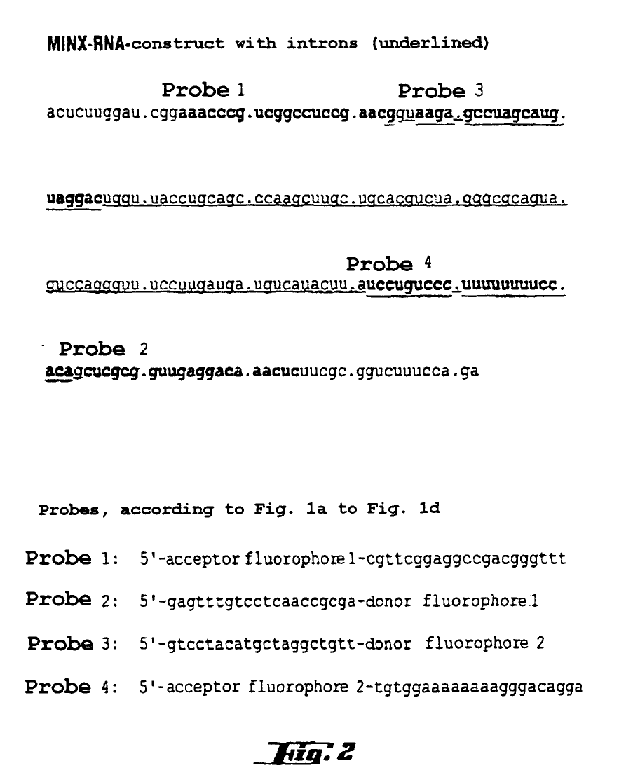

As already described in more detail, the spliceosome opens in the first splicing step the linkage between exon 1 and intron at the 5′ splicing site of the intron. Only in the second splicing step are exon 1 and exon 2 covalently linked. As a result, exon 1 is no longer linked to the mRNA during the first step of the splicing reaction and is thus removable from the splicing reaction. In connection with constructs to which, for example, corresponding fluorophore pairs on oligonucleotides bind in the intron and in exon 2, it is therefore possible to make a statement on whether, for example, an inhibition has occurred in the first splicing step. If, for example, two different fluorophore pairs which emit at different wavelengths are incorporated at the 5′ end of exon 1 and into the intron and at the 3′ end of the intron and the 5′ end of exon 2 of the pre-mRNA, then it is possible to follow both the first splicing step and the second splicing step in a test system. Examples of suitable nucleic acid constructs are disclosed in the German patent application 199 09 156. FIG. 2 shows a principal arrangement for implementation. Depending on the signal sequence, the use of different wavelengths can indicate the course of the splicing process. The invention therefore also relates to a method of studying the splicing process.

For studies in the yeast system it is possible, for example, to start from the pre-mRNA for yeast U3 (Mougin, A. et al. (1996), RNA, 2: 1079-93).

The studies of the individual splicing reactions with the aid of a test system of the invention are commonly carried out by using a composition comprising the individual splicing components, preferably small nuclear ribonucleoprotein particle (snRNP) components and non-snRNP components. The snRNP components particularly comprise U1, U2, U4, U5 and/or U6 proteins. Preference is given in particular to using appropriate cell extracts, in particular eukaryotic cell extracts, for the studies. It is possible, for example, to obtain the cell extracts from animal cells, in particular mammalian cells, especially Hela cells, in particular from nuclear extracts of HeLa cells or cell extracts of fungi, in particular yeasts, according to methods generally known to the skilled worker (see examples). The cell extracts generally comprise all important factors in order to be able to carry out splicing in vitro.

It is essential for carrying out the studies to use further aids such as, for example, buffer solutions, stabilizers and/or energy equivalents, in particular ATP.

The present invention therefore also relates to a method for preparing a test system in which at least one mobile spliceable nucleic acid together with associated probes comprising corresponding fluorophores and at least one gel-free detection system and also, where appropriate, at least one composition comprising splicing components and, where appropriate, further aids are combined. Preferred embodiments of the individual components have already been described in more detail.

The present invention therefore further relates to a method for finding an active substance, which comprises

(a) incubating one or more identical or different mobile nucleic acid(s) with at least one spliceable nucleic acid sequence together with associated probes with corresponding fluorophores in the presence of at least one substance to be studied and at least one composition comprising splicing components and, where appropriate, in the presence of further aids under suitable conditions, and

(b) detecting the splicing product which may have formed by means of a gel-free detection system.

Preferred individual components of the method of the invention have already been described in more detail above.

The active substance here may be a pharmaceutically active compound, a natural substance in the broadest sense, a fungicide, a herbicide, a pesticide and/or an insecticide, and is preferably an antibiotic. The substance to be studied is generally a naturally occurring, a naturally occurring and chemically modified, and/or a synthetic substance. The method of the invention makes it possible in particular to screen “combinatorial substance libraries” in a simple and rapid manner.

In the introduction of the description it was already indicated that various disorders can be attributed to a disruption of the splicing mechanism. The present invention is therefore also suitable for diagnosing a disorder. The present invention therefore further relates to a method for diagnosing a disorder, which comprises

(a) incubating one or more identical or different mobile nucleic acid(s) preferably with at least one spliceable nucleic acid together with probes with associated corresponding fluorophores in the presence of at least one substance to be studied and at least one composition comprising splicing components and, where appropriate, further aids under suitable conditions, and

(b) detecting the splicing product which may have formed by means of a gel-free detection system.

Very preferably the substance is a nucleic acid, preferably RNA.

The disorders to be diagnosed here are preferably genetic disorders, cancers and/or viral diseases, in particular Graves' disease, spinal muscular atrophy, β′ thalassemia, cancers related to the c-erb oncogene, hepatitis C infections and/or Herpes simplex virus infections. In particular, the present invention can identify truncated alternative splicing products of the mutated APC oncogene directly and thus is an alternative to the previously used PTT (protein truncation test) (Bala, S., et al., (1996) Hum Genet 98 (5): 528-533).

The following figures and examples are intended to describe the invention in more detail without restricting it.

### EXAMPLES

1. The RNA Construct:

The mRNA to be spliced comprises at least two exons which are separated by an intron which may be of either natural or molecular biological origin.

2. Preparation of Nuclear Extracts:

2.1 Nuclear Extracts from Mammalian Cells

Nuclear extracts are prepared from mammalian cells by using cell cultures of Hela cells. To this end, the cells are sedimented from the culture medium by centrifugation (1 000×g, 10 min) and washed with phosphate buffer. The cell sediment is then taken up in five volumes of buffer A (10 mM HEPES, 1.5 mM MgCl₂, 10 mM KCl, 0.5 mM DTT, pH 7.9, 4° C.) and incubated for 10 minutes. The cells are again sedimented and taken up in two volumes of buffer A. This suspension is disrupted using a Dounce homogenizer (pestle B) (moving the pestle up and down 10 times). The nuclei are sedimented by centrifugation. Finally, the nuclei are again taken up in buffer A and centrifuged at 25 000×g for 20 minutes. The sediment is taken up in 3 ml of buffer B (20 mM HEPES, 25% (v/v) glycerol, 0.42 M NaCl, 1.5 mM MgCl₂, 0.2 mM EDTA, 0.5 mM phenylmethylsulfonyl fluoride (PMSF), 0.5 mM DTT, pH 7.9) and disrupted again using the Dounce homogenizer. The suspension forming is incubated on a magnetic stirrer for 30 minutes and then centrifuged at 25 000×g for 30 minutes. This is again followed by centrifugation at 25 000×g (30 min). The clear supernatant is dialyzed against 50 volumes of buffer C (20 mM HEPES, 20% (v/v) glycerol, 0.1 M KCl, 0.2 mM EDTA, 0.5 mM PMSF, 0.5 mM DTT, pH 7.9). The dialyzate is centrifuged (25 000×g, 20 min) and the resulting supernatant can be stored as nuclear extract in liquid nitrogen (Dignam; J. D. et al. (1983) Nucleic Acid Res., 11,1475).

2.2 Cell Extracts from Yeast Cells

Cell extracts from yeast cells are prepared in a very similar way. Yeast cells of a protease-deficient strain (BJ926, EJ101 or similar strains) are sedimented by centrifugation (1 500×g, 5 min, 4° C.) in the logarithmic growth phase. The cells are resuspended in two to four volumes of ice cold water and again centrifuged at 1 500×g (5 min, 4° C.). The cells are then taken up in one volume of zymolyase buffer (50 mM Tris HCL, 10 mM MgCl₂, 1 M sorbitol, 30 mM DTT, pH 7.5) and incubated at room temperature for 30 minutes. The cells are removed by centrifugation (1 500×g, 5 min, 4° C.), taken up in three volumes of zymolyase buffer with 2 mg (200 U) of zymolyase 100 T and incubated on a shaker (50 rpm) at 30° C. for 40 minutes. The spheroblasts formed are removed by centrifugation (1 500×g, 5 min, 4° C.) and washed once in 2 ml of ice cold zymolyase buffer. The sediment is washed with two volumes of lysis buffer (50 mM Tris HCl, 10 mM MgSO₄, 1 mM EDTA, 10 mM potassium acetate, 1 mM DTT, protease inhibitors, 1 mM PMSF, pH 7.5) and finally taken up in one volume of lysis buffer. The spheroblasts are then lysed in a Dounce homogenizer by moving the pestle up and down 15 to 20 times (distance 1-2 μm). The same volume of extraction buffer (lysis buffer +0.8 M ammonium sulfate, 20% (v/v) glycerol) is added to the lysate and the mixture is incubated on an end-over-end shaker for 15-30 minutes (4° C.). This is followed by centrifugation at 100 000×g for 90 minutes (4° C.). The supernatent is dialyzed against one hundred volumes of storage buffer (20 mM Tris HCl, 0.1 mM EDTA, 10% (v/v) glycerol, 100 mM KCl, 1 mM DTT, protease inhibitors, 1 mM PMSF, pH 7.5). The dialyzate is removed by centrifugation at 10 000×g (4° C.) and the supernatant is stored in liquid nitrogen (Dunn, B. & Wobbe, C. R. (1994) Preparation of protein extracts from yeast, In: Ausubel, F. M. et al. (eds.) Current Protocols in Molecular Biolody, 2nd Volume, John Wiley and Sons, Inc., USA, pp. 13.13.1-13.13.9).

3. In Vitro Splicing of Constructs in a Test System

Excising the intron from the RNA sequence leads at the boundary of the two now linked exons to a nucleotide sequence which is not present in the unspliced pre-mRNA. Complementary nucleotide sequences which hybridize to the 3′ terminus of exon 1 and to the 5′ terminus of exon 2 now form a continuous sequence. The proximity of two corresponding fluorophores, which results therefrom, makes a fluorescence resonance energy transfer (FRET) possible. The signal measured after excitation at a specific extinction wavelength indicates the successfully concluded splicing process. The assay is analyzed in a suitable analyzer (fluorescence spectrometer, Perkin Elmer, Uberlingen, Germany; fluorescence reader, Fluostar Galaxy, BMG, Offenburg, Germany; Light Cycler, Roche, Mannheim, Germany).

All experiments described were carried out according to standard methods, as are described in (Eperon, I. C. and Krainer, A. R. (1994) Splicing of mRNA precursors in mammalian cells. In RNA Processing, vol. I—A Practical Approach (B. D. Hames and S. J. Higgins, eds.) Oxford: IRL Press, pp. 57-101).

3.1 In Vitro Transcription Procedure

The construct described in FIG. 2 was provided with a T7 promoter by means of a standard PCR reaction and amplified. It was then transcribed from the plasmid coding therefor into the corresponding mRNA by means of in vitro transcription. Likewise, the corresponding construct without the intron was used in the experiments as a positive control and for subsequent comparison.

The reaction was carried out under the following conditions:

5 μl of 5×transcription buffer (200 mM Tris-HCl pH 7.9, 30 mM MgCl₂, 10 mM spermidine, 50 mM NaCl)

0.5 μl of RNAsin (40 U/μl)

1 μl of DTT (100 mM)

1 μl of NTPs (ATP, GTP, CTP and UTP at 5 mM)

1 μl of MINX template DNA (1 mg/ml)

1 μl T7 polymerase (10 U/μl)

ad 10 μl with H₂O

The transcription mixture was incubated at 37° C. for 2 h and then purified via a preparative urea gel according to standard methods. The labeled RNA was found by placing the gel on a thin layer chromatography plate carrying a 0.25 mm thick layer of silica gel 60 with fluorescent indicator UV254 (Macherny-Nagel) and irradiated from above using a UV lamp with a wavelength of 254 nm. The RNA band absorbs the wavelength required for exciting the fluorescence indicator and thus results in a region on the thin layer chromatography plate, which is not excited and becomes visible as a shadow. On the basis of this shadow, the RNA was excised using a scalpel. The gel fragment was cut and the RNA was extracted from the gel at 4° C. overnight using elution buffer (500 mM Na acetate pH 5, 1 mM EDTA pH 8, 2.5% phenol/chloroform). The acrylamide was then sedimented bei centrifugation at 13,000×g for 10 minutes and the RNA was precipitated from the supernatant with ethanol/sodium acetate, washed, dried and taken up in 10 mM Tris-HCl, pH 7.5.

3.2 Splicing Reaction Procedure

7 μl of HeLa cell nuclear extract (=35% v/v) were incubated with 3.25 mM MgCl₂, 35 mM KCl, 2 mM ATP, 20 mM phosphocreatine, 1 U/μl RNAsin and 1 μg of MINX pre-mRNA (Zillmann et al., 1988, Molecular and Cellular Biology, 8: 814) in a reaction volume of 20 μl at 30° C. for 0, 10, 20, 30 and 40 minutes. Pre-mRNA which did not contain the intron was used for comparison. The reactions were then stopped by adding 400 μl of proteinase K buffer (100 mM Tris-HCl, pH 7.5, 12.5 mM EDTA, 150 mM NaCl, 1% SDS, 0.1 mg proteinase K), followed by adding in each case 50 pmol of probe and measuring the fluorescence resonance energy transfer in a fluorescence spectrophotometer (Perkin Elmer).

The splicing process was followed in time (online detection in real time) by pipetting the reaction mixture as above, including the probes, and monitoring the increase and decrease, respectively, of the signal by means of a fluorescence reader over a period of 40 minutes.

A large number of small probes can be detected in the Light Cycler System (Roche, Switzerland) or in a fluorescence reader (Fluostar Galaxy, BMG, Offenburg, Germany), for example to diagnose the truncated splicing products of the APC oncogene.

4. Preparation of the Probes

The oligonucleotides used can be prepared by a standard solid-phase DNA synthesis using a 392 RNA/DNA synthesizer (Applied Biosystems, 1992: Evaluating and Isolating Synthetic Oligonucleotides). The syntheses are carried out on a scale of 0.2 or 1 μmol according to the standard cycles recommended by Applied Biosystems, maintaining the 5′-terminal dimethoxytrityl group (“trityl-on”). For the synthesis of a 3′-labeled probe, a fluorophore-loaded support (controlled pore glass, CPG, support) is used. Thereby the fluorophore (fluorescein-CPG, TAMRA-CPG, Glen Research; Sterling, Va.) is incorporated at the 3′ terminus already during synthesis. A 5′-labeled probe is prepared by a standard oligosynthesis, and the 5′ modification is obtained by reacting the 5′ end with a fluorescein phosphoramidite (Glen Research).

The oligonucleotides are removed from the solid phase by cleaving with an aqueous ammonia solution (33%) at room temperature over a period of 1 h. The protective groups are then cleaved off by adding again aqueous ammonia solution (33%) and subsequent incubation at 55° C. for 6 h. The efficiency of the individual coupling steps is determined via UV/VIS spectrometry by measuring the absorption at 495 nm of the trityl groups removed during synthesis. The deprotected oligonucleotide solutions are frozen in liquid nitrogen and lyophilized, controlling the pH every 20 minutes and adding, where appropriate, triethylamine (10 μl) in order to avoid detritylation at the terminal 5′ position.

The lyophilisate is then taken up in 400 μl of 100 mM triethylammonium acetate, pH 7.0 and the oligonucleotides are purified by means of reversed phase HPLC on a Hypersil reversed phase C-18 column with a flow rate of 1 ml/min. The following gradient is used for elution: 11-26% 70% acetonitrile/30% 100 mM triethylammonium acetate, pH 7.0 (0-20 min), 26-44% 70% acetonitrile/30% 100 mM triethylammonium acetate, pH 7.0 (20-30 min), 44-100% 70% acetonitrile/30% 100 mM triethylammonium acetate, pH 7.0 (30-33 min). The chromatography is monitored by UV spectroscopy at wavelengths λ=260 and 290 nm. The product fractions are combined, lyophilized and traces of triethylammonium acetate are removed by taking up the lyophilisate several times in water (in each case 1 ml) and subsequent lyophilization. The lyophilisate is taken up in water (500 μl) and the oligonucleotide concentration is determined via UV absorption at a wavelength of λ=260. The 5′-terminal trityl group is removed (“detritylation”) by cleaving with aqueous acetic acid (80% strength, 10 μl/nmol of oligonucleotide) at room temperature for 20 min. After adding aqueous sodium acetate solution (3M, pH 5.2, 1.5 μl/mol of oligonucleotide) and isopropanol (34 μl/nmol of oligonucleotide), the detritylated oligonucleotides are precipitated at −80° C. for 10 min. The oligonucleotides are removed by centrifugation (17 000 g) at 4° C., the supernatant is discarded and the white milky precipitate obtained is washed with aqueous ethanol solution (2×200 μl, 70% strength), cooled to −20° C. The oligonucleotides are again centrifuged (1 7 000×g) at 4° C., the supernatant is discarded and the precipitate obtained is dried at room temperature in a high vacuum. The resulting lyophilisate was taken up in water (500 μl) and the pH was adjusted to pH 7.0 with aqueous sodium hydroxide solution.

The purity of the detritylated oligonucleotides obtained was determined by reversed phase HPLC using an ODA Hypersil reversed phase C-18 column. The following gradient was used for elution: 5-25% 70% acetonitrile/30% 100 mM triethylammonium acetate, pH 7.0 (0-20 min), 25-45% 70% acetonitrile/30% 100 mM triethylammonium acetate, pH 7.0 (20-25 min), 45-100% 70% acetonitrile/30% 100 mM triethylammonium acetate, pH 7.0 (25-28 min).

For desalting, the oligonucleotides are applied to a gel filtration column (NAP-5) preequilibrated with water and are eluted with 1.5 ml of water. The collected fractions (100 μl) are determined via UV absorption at a wavelength of λ=260, the main fractions are combined, the volume is reduced by lyophilization and the oligonucleotide concentration is determined by UV spectrometry.

1. A homogeneous test system comprising
(a) at least one or more identical or different mobile nucleic acid(s) having at least one spliceable nucleic acid comprising at least one intron,
(b) at least two probes, comprising corresponding fluorophores which preferably bind to the 5′ and/or 3′ ends of an exon/intron,
(c) at least one gel-free detection system for detecting a splicing reaction, where appropriate
(d) at least one composition comprising splicing components,
(e) further aids.

2. The test system as claimed in claim 1, wherein the spliceable nucleic acid comprises at least two exons which are separated by at least one intron.

3. The test system as claimed in either of claims 1 and 2, wherein the at least two probes comprise nucleic acids which are complementary to partial sequences of at least two exons and/or to partial sequences of at least one exon and one intron of the spliceable nucleic acid according to feature (a), the partial sequences being attached to and/or removed from one another by the splicing reaction.

4. The test system as claimed in any of claims 1-3, wherein the spliceable nucleic acid and the probe-binding nucleic acid sequence are linked to one another.

5. The test system as claimed in any of claims 1-4, wherein the probes according to feature (b) hybridize to the spliceable nucleic acid according to (a).

6. The test system as claimed in any of claims 1-5, wherein the corresponding fluorophores are selected from fluorescein/rhodamine and derivatives therefrom such as carboxyfluorescein succinimidyl ester/Texas Red sulfonyl chloride, FITC/TMR (fluorescein-5-isothiocyanate/tetramethylrhodamine, and naphthalene/dansyl, dansyl/DDPM (N-[4-dimethylamino)-3,5-dinitrophenyl]maleimide), IAEDANS/DiO-C14 (5-(2-((iodoacetyl)amino)ethyl)amino)naphthalene-1-sulfonic acid/3,3-ditetradodecyloxacarbocyanine), ANAI/IPM (2-anthracene-N-acetylimidazole/3-(4-isothiocyanatophenyl)-7-diethyl-4-amino-4-methylcoumarin, BPE/CY5 (B-phycoerythrin/carboxymethyl indocyanine N-hydroxysuccimidyl ester) and lanthanide chelates.

7. The test system as claimed in any of claims 1-6, which comprises corresponding fluorophores which are accessible to time-resolved fluorescence resonance energy transfer.

8. The test system as claimed in any of claims 1-7, wherein at least one nucleic acid is an RNA.

9. The test system as claimed in any of claims 1-8, wherein the composition according to feature (c) comprise small nuclear ribonucloprotein particle (snRNP) components and non-snRNP components.

10. The test system as claimed in claim 9, wherein the snRNP components comprise U1, U2, U4, U5 and/or U6 proteins.

11. The test system as claimed in any of claims 1-10, wherein the composition according to feature (c) is a cell extract, in particular a eukaryotic cell extract or nuclear extract.

12. The test system as claimed in claim 11, wherein the nuclear extract is obtained from animal cells, in particular mammalian cells, or from fungi, in particular yeasts.

13. The test system as claimed in any of claims 1 to 12, wherein the further aids are selected from buffer solutions, stabilizers and/or energy equivalents, in particular ATP.

14. A method for preparing a test system as claimed in any of claims 1 to 13, which comprises combining at least one mobile spliceable nucleic acid together with associated probes comprising corresponding fluorophores and at least one gel-free detection system and also, where appropriate, at least one composition comprising splicing components and, where appropriate, further aids.

15. A method for finding an active substance, which comprises
(a) incubating one or more identical or different mobile nucleic acid(s) with at least one spliceable nucleic acid sequence together with associated probes with corresponding fluorophores in the presence of at least one substance to be studied and at least one composition comprising splicing components and, where appropriate, in the presence of further aids under suitable conditions, and
(b) detecting the splicing product which may have formed by means of a gel-free detection system.

16. The method as claimed in claim 15, wherein the active substance is selected from natural substances in the broadest sense, herbicides, insecticides, pesticides, antibiotics, pharmaceuticals, combinatorial substance libraries.

17. The method as claimed in claim 15 or 16, wherein the substance to be studied is selected from a naturally occurring, naturally occurring and chemically modified, and/or synthetic substance.

18. A method for diagnosing a disorder, which comprises
(a) incubating one or more identical or different mobile nucleic acid(s) with at least one spliceable nucleic acid sequence together with associated probes with corresponding fluorophores in the presence of at least one substance to be studied and preferably at least one composition comprising splicing components and, where appropriate, in the presence of further aids under suitable conditions, and
(b) detecting the splicing product which may have formed by means of a gel-free detection system.

19. The method as claimed in claim 18, wherein the substance is a nucleic acid, preferably RNA.

20. The method as claimed in claim 18, wherein the disorder is a genetic disorder, a cancer and/or a viral disease.

21. The method as claimed in any of claims 18-20, wherein the disorder is selected from Graves' disease, spinal muscular atrophy, β′ thalassemia, cancers related to the c-erb oncogene, the APC oncogene, hepatitis C infection and/or Herpes simplex virus infection.


SEQUENCE LISTING (5 sequences)

SEQ ID NO: 1 - RNA, 192 nt, Homo sapiens
acucuuggaucggaaacccgucggccuccgaacgguaagagccuagcauguaggacugguuaccugcagcccaagcuugcugcacgucuagggcgcaguaguccaggguuuccuugaugaugucauacuuauccugucccuuuuuuuuccacagcucgcgguugaggacaaacucuucgcggucuuuccaga

SEQ ID NO: 2 - DNA, 21 bp, Homo sapiens
cgttcggaggccgacgggttt

SEQ ID NO: 3 - DNA, 20 bp, Homo sapiens
gagtttgtcctcaaccgcga

SEQ ID NO: 4 - DNA, 20 bp, Homo sapiens
gtcctacatgctaggctgtt

SEQ ID NO: 5 - DNA, 22 bp, Homo sapiens
tgtggaaaaaaaagggacagga